(19) United States
(12) Patent Application Publication
(10) Pub. No.: US 2006/0070878 A1
(43) Pub. Date: Apr. 6, 2006

(54) Electrochemical biosensor strip

(76) Inventors: Shu-Mei Wu, Taipei City (TW); Chia-Chi Wu, Kaohsiung City (TW); Yeong-Dar Chen, Kaohsiung City (TW); Chao-Wang Chen, Taipei City (TW)

Correspondence Address: HDSL, 4331 STEVENS BATTLE LANE, FAIRFAX, VA, 22033, US

(21) Appl. No.: 10/958,291

(22) Filed: Oct. 6, 2004

(51) Int. Cl. G01N 33/487; G01N 27/26

(52) U.S. Cl. 204/403.01; 204/412

(57) ABSTRACT

An electrochemical biosensor strip has a base, an electrode system, a spacer and a cover. The electrode system is laid on the base and preferably comprises at least three electrodes. There is a short circuit formed between two selected electrodes of the at least three electrodes. The spacer is laid on the electrode system and exposes a portion of the electrode system for electrical connection with a meter and a different portion of the electrode system for application of a test reagent to those exposed surfaces of electrode system. The test reagent is a reagent that is specific for the test to be performed by the strip. The cover is covered on the spacer. The short circuit is used to switch on a meter for increasing the operative convenience and further used to recognize the suitable meter. The electrochemical biosensor strip may have a rough unit formed on the base for increasing the adhesion of the test reagent for the test to be performed by the strip.

### BACKGROUND OF THE INVENTION

1. Field of the Invention

The present invention relates in general to an electrochemical biosensor strip. More particularly, the present invention relates to an electrochemical biosensor strip that can switch on a biosensor when the electrochemical biosensor strip is inserted into the biosensor or can increase a test reagent that is specific for the test to be performed by the strip laid on the strip with high test accuracy.

2. Description of the Related Art

Since the improvement of the science and technology, many tests can be operated by users in house. In the market, many disposable strips are used for measuring specific components in a biological fluid and can be operated by users in house. Analytical biosensor strips are useful in chemistry and medicine to determine the presence and concentration of a biological analyte. Such strips are needed, for example, to monitor glucose in diabetic patients and lactate during critical care events. Biosensor strips are used in the chemical industry, for example, to analyze complex mixtures. They are also used in the food industry and in the biochemical engineering industry. Biosensor strips are also useful in medical research or in external testing. In external testing, they can function in a non-invasive manner (i.e., where they come into contact with blood withdrawn by a syringe or a pricking device).

A conventional electrochemical biosensor strip has a base, an electrode system, an insulating substrate, a test reagent and a cover. The electrode system is laid on the base and comprises two electrodes separated from each other. The insulating substrate is laid down onto the electrode system and has a first opening and a second opening. The first opening exposes portions of the electrode system for electrical connection with a meter, which measures some electrical property of a test sample after the test sample is mixed with the test reagent of the strip. The second opening exposes a different portion of the electrode system for application of the test reagent to those exposed surfaces of electrode system. The test reagent is a reagent that is specific for the test to be performed by the strip. The test reagent may be applied to the entire exposed surface area of the electrode system in the area defined by the second opening. The cover is covered on the electrode system and the test reagent to protect the test reagent.

However, it is inconvenient to turn on the meter before using the meter by users and then inserting the strip into the meter to test the specific component in the test sample. Furthermore, since different manufacturing factories design different meters with different structures, strips designed suitable with the corresponding meters also have different structures. In addition, different meters may have different parameters for measuring the specific component in the biological fluid. If a wrong strip is inserted into an unsuitable meter, a test result may be incorrect. Furthermore, since the base is prepared by polyethylene terephthalate (PET) or polyvinyl chloride (PVC) which is hydrophobic, the test reagent laid on the base is unstable. The strip is manufactured by a sheet of the PET or PVC substance and the sheet is cut for forming multiple strips after every needed substances are laid on the sheet. When the cutting step is proceeding, the test reagent is easy to shake off from the base.

### SUMMARY OF THE INVENTION

In one aspect of the present invention, an electrochemical biosensor strip in accordance with the present invention has a structure for switch on a meter and also may be used to identify a suitable meter.

Accordingly, the electrochemical biosensor strip comprises a base, an electrode system, a spacer, a test reagent and a cover. The electrode system is laid on the base and preferably comprises at least three electrodes. There is a short circuit formed between two selected electrodes of the at least three electrodes. The spacer is laid on the electrode system and has an opening. The spacer exposes a portion of the electrode system for electrical connection with a meter, which measures some electrical property of a test sample after the test sample is mixed with the test reagent of the strip. The opening exposes a predetermined portion of the electrode system for applying of the test reagent to those exposed surfaces of electrode system. The test reagent is a reagent that is specific for the test to be performed by the strip. The test reagent may be applied to the entire exposed surface area of the electrode system in the area defined by the opening. The cover is covered on the spacer to protect the test reagent. The short circuit can be used to switch on the meter.

In another aspect of the present invention, an electrochemical biosensor strip in accordance with the present invention has a rough unit for increasing the adhesion of the test reagent for the test to be performed by the strip. Furthermore, the rough unit can prevent the test reagent shaking off from the base and increase the flow rate of the test sample into the opening of the spacer.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a perspective view of a first embodiment of an electrochemical biosensor strip in accordance with the present invention;

FIG. 2 is an exploded perspective view of the electrochemical biosensor strip of FIG. 1;

FIG. 3 is a top view of a second embodiment of a carbon layer of an electrode system of the electrochemical biosensor strip of FIG. 1;

FIG. 4 is a top view of a third embodiment of the carbon layer of the electrode system of the electrochemical biosensor strip of FIG. 1;

FIG. 5 is a top view of a fourth embodiment of the carbon layer of the electrode system of the electrochemical biosensor strip of FIG. 1;

FIG. 6 is a top view of a fifth embodiment of the carbon layer of the electrode system of the electrochemical biosensor strip of FIG. 1;

FIG. 7 is a top view of a sixth embodiment of the carbon layer of the electrode system of the electrochemical biosensor strip of FIG. 1;

FIG. 8 is an exploded perspective view of a second embodiment of an electrochemical biosensor strip in accordance with the present invention;

FIG. 9 is a partial perspective view of the electrochemical biosensor strip of FIG. 8;

FIG. 10 is a perspective view of a third embodiment of an electrochemical biosensor strip in accordance with the present invention;

FIG. 11 is an exploded perspective view of the electrochemical biosensor strip of FIG. 10; and

FIG. 12 is an exploded perspective view of a fourth embodiment of an electrochemical biosensor strip in accordance with the present invention.

### DESCRIPTION OF THE PREFERRED EMBODIMENTS

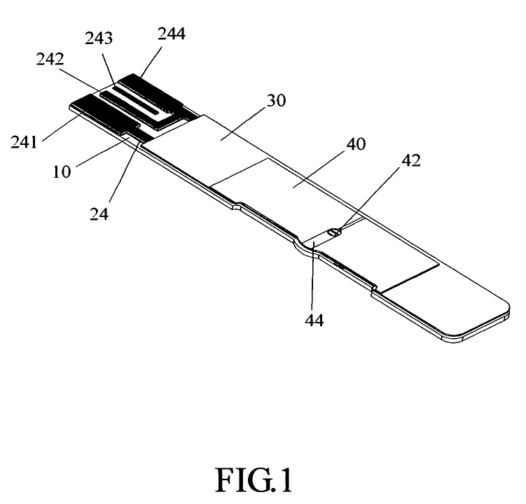

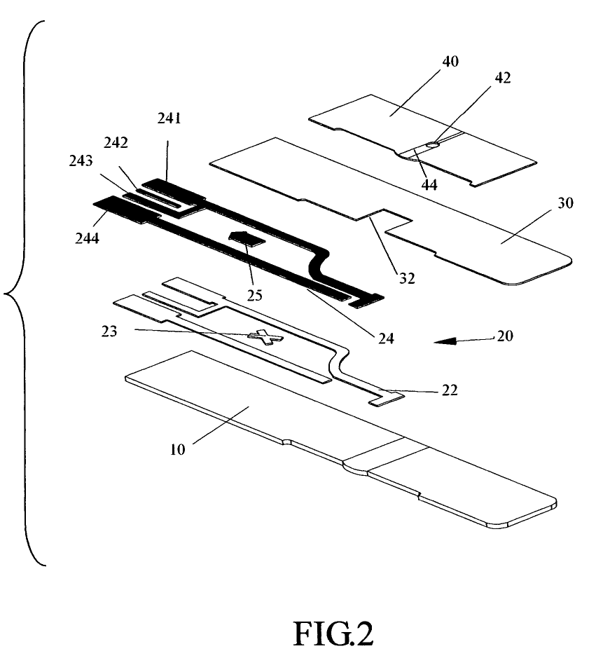

With reference to FIGS. 1 and 2, a first embodiment of an electrochemical biosensor strip in accordance with the present invention comprises a base (10), an electrode system (20), a spacer (30), a test reagent and a cover (40).

The base (10) may be rectangular and preferably is an insulating substance. The electrode system (20) is laid on the base (10) and preferably comprises two layers that are a silver layer (22) and a carbon layer (24). The silver layer (22) is laid on the base (10) and the carbon layer (24) is laid on the silver layer (22). The carbon layer (24) and the silver layer (22) respectively comprise at least three electrodes that respectively have a first end and a second end. In the drawings, the silver layer (22) has three electrodes and the carbon layer (24) has four electrodes that respectively named a first electrode (241), a second electrode (242), a third electrode (243) and a fourth electrode (244). There is a short circuit formed between two electrodes of the carbon layer (24) and the corresponding electrodes of the silver layer (22). Other electrodes are separated so that the electrodes do not interfere with the electrochemical events at the other electrode. In the drawings, there is a short circuit formed between the second electrode (242) and the fourth electrode (244). Preferably, the one electrode of the electrodes that formed the short circuit is a reference electrode. The short circuit is used to switch on a suitable meter.

The first ends of the electrodes are parallel and formed along a long edge of the strip. The second ends of the electrodes are defined corresponding to the test reagent. Preferably, the silver layer (22) further has a forked shape unit (23) formed between the electrodes. More preferably, the carbon layer (24) further has an arrowhead shape unit (25) formed on the forked shape unit (23) of the silver layer (22). When users are inserting wrong direction of the strip, the users will see the forked shape unit (23) for reminding the users that the direction is wrong. On the contrary, the users will see the arrowhead shape unit (25) when the inserting direction is correct.

The spacer (30) is covered on the electrode system (20) and comprises an opening (32). The electrodes will be exposed as the spacer (30) is covered on the electrode system (20) for electrical connection with a meter which measures some electrical property of a test sample after the test sample is mixed with the test reagent of the strip. The opening (32) exposes a predetermined portion of the electrode system (20) for application of the test reagent to the exposed surface of electrode system (20). The opening (32) is preferably formed in a side of the spacer (30).

The test reagent is a reagent that is specific for the test to be performed by the strip. The test reagent may be applied to the entire exposed surface area of the electrode system (20) in the area defined by the opening (32). The cover (40) is covered on the spacer (30) to protect the test reagent and preferably has a hole (42) formed corresponding to the opening (32) of the spacer (30). Preferably, the cover (40) further has a channel (44) opening to the opening (32) and communicated with the hole (42). The channel (44) and the hole (42) are both used to increase the flow rate of an analyte-containing fluid received in the opening (32). When an analyte-containing fluid, for example, a drop of blood, has been received in the opening (32) of the strip, the analyte reacts with the test reagent and an output signal corresponding to a sensing current is generated and is detected by a meter.

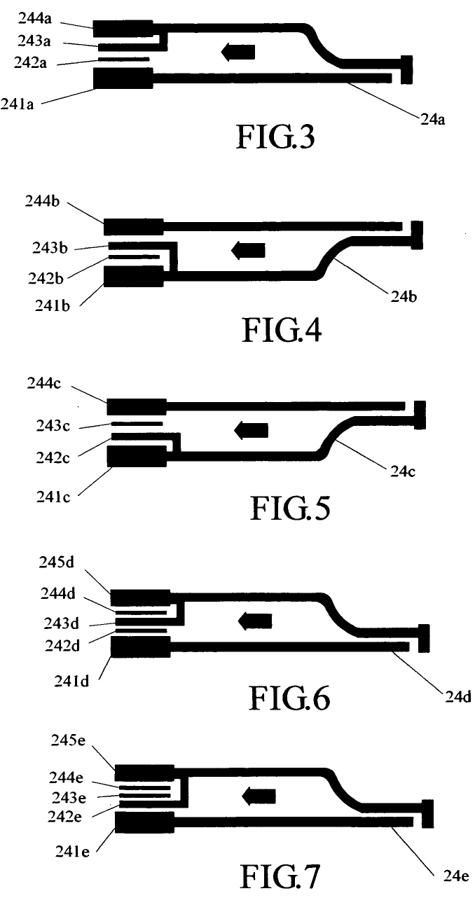

With reference to FIG. 3, a second embodiment of the carbon layer (24a) in accordance with the present invention has four electrodes that respectively named a first electrode (241a), a second electrode (242a), a third electrode (243a) and a fourth electrode (244a). There is a short circuit between the third electrode (243a) and the fourth electrode (244a). With reference to FIG. 4, a third embodiment of the carbon layer (24b) in accordance with the present invention has four electrodes that respectively named a first electrode (241b), a second electrode (242b), a third electrode (243b) and a fourth electrode (244b). There is a short circuit between the first electrode (241b) and the third electrode (243b). With reference to FIG. 5, a fourth embodiment of the carbon layer (24c) in accordance with the present invention has four electrodes that respectively named a first electrode (241c), a second electrode (242c), a third electrode (243c) and a fourth electrode (244c). There is a short circuit between the first electrode (241c) and the second electrode (242c).

With further reference to FIG. 6, a fifth embodiment of the carbon layer (24d) in accordance with the present invention has five electrodes that respectively named a first electrode (241d), a second electrode (242d), a third electrode (243d), a fourth electrode (244d) and a fifth electrode (245d). There is a short circuit between the third electrode (243d) and the fifth electrode (245d). With reference to FIG. 7, a sixth embodiment of the carbon layer (24e) in accordance with the present invention has five electrodes that respectively named a first electrode (241e), a second electrode (242e), a third electrode (243e), a fourth electrode (244e) and a fifth electrode (245e). There is a short circuit between the second electrode (242e) and the fifth electrode (245e).

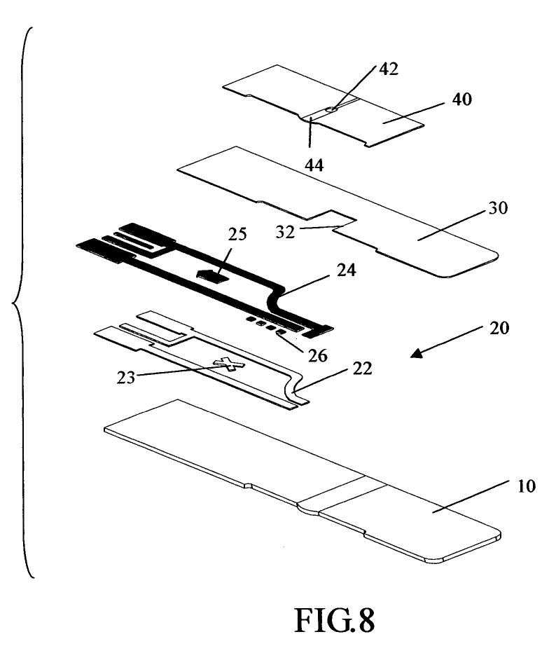

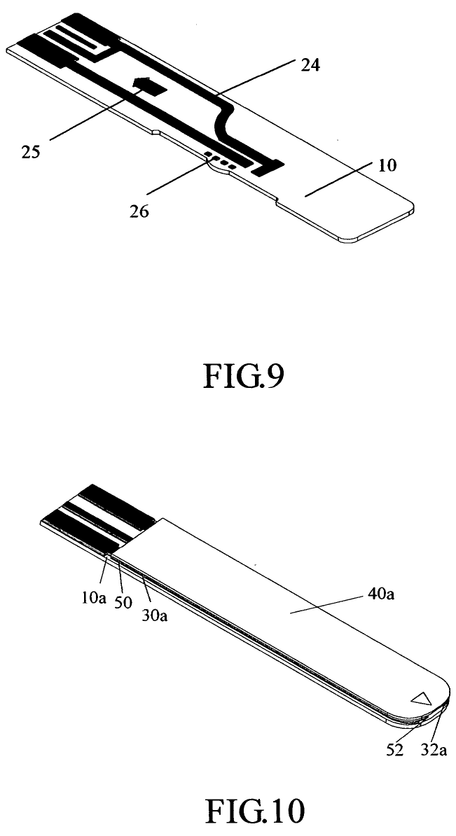

With reference to FIGS. 8 and 9, a second embodiment of the electrochemical biosensor strip in accordance with the present invention further comprises a base (10), an electrode system (20), a spacer (30), a test reagent and a cover (40) that the same with the first embodiment of the electrochemical biosensor strip and further has a rough unit (26) laid on the base (10) and located corresponding to the test reagent. Preferably, the rough unit (26) is a line or multiple lines. The rough unit (26) is preferably laid on an outside of the second end of the electrodes and adjacent to the electrodes. The rough unit (26) is preferably prepared by electric conduction substance or non-electric conduction substance. More preferably, the rough unit (26) is prepared by carbon and separated from the electrode system (20). For decreasing manufacturing cost, the rough unit (26) is printed by carbon when the carbon layer (24) of the electrode system (20) is printed. The rough unit (26) can increase the rough of the base (10). The test reagent laid on the base (10) will not easy to shake off when the strip is cut for separating. Furthermore, the rough unit (26) has a height for blocking the test reagent to lose from the base (10). In addition, the rough unit (26) is located on the outside of the electrode system (20) so the rough unit (26) can form a wall for protection the test reagent from falling.

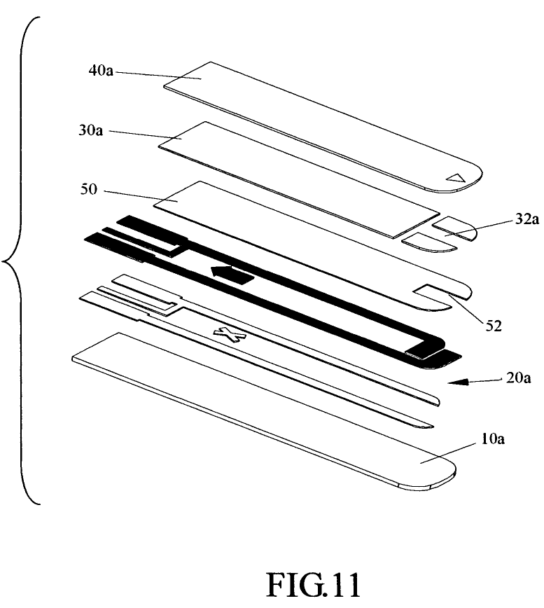

With reference to FIGS. 10 and 11, a third embodiment of the electrochemical biosensor strip in accordance with the present invention comprises a base (10a), an electrode system (20a), an insulating substance (50), a spacer (30a) and a cover (40a). The spacer (30a) comprises an opening (32a) formed in an end of the spacer (30a). Preferably, the opening (32a) is T shape. The opening (32a) is used for introducing a sample into the strip. The insulating substance (50) is laid on the base (10a) and has an opening (52) formed corresponding to the opening (32a). The test reagent is laid on the opening (52) and the opening (32a). The electrode system (20a) has three electrodes and there is a short circuit between two electrodes of the three electrodes.

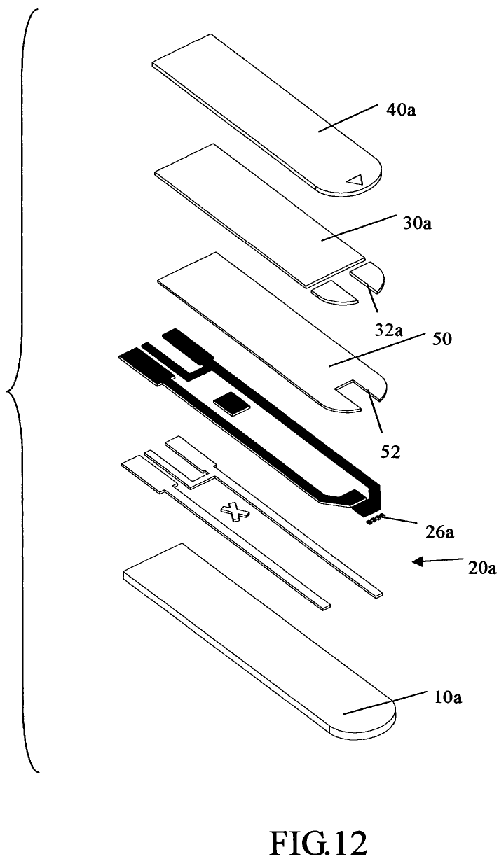

With further reference to FIG. 12, a fourth embodiment of the electrochemical biosensor strip in accordance with the present invention further comprises a rough unit (26a) formed on the base (10a). The rough unit (26a) is formed on one end of the base (10a). In the drawing, the rough unit (26a) is parallel with one end of the electrode and near an outside of the base (10a) corresponding to the test reagent. Preferably, the rough unit (26a) is formed adjacent to the electrode.

In use, because of the users do not need to open the meter it is advantageous to increase useful convenience of using the strip. When users are inserting the strip into the meter, the short circuit structure of the strip will switch on the meter which is designed with the strip. When the strip switched on the meter indicates that the strip is correct to the corresponding meter. This step will increase test accuracy for preventing some parameters in the meter is wrong so that the test result will not correct. It will save time and operate simply to switch on a meter by inserting the strip.

Furthermore, the rough unit can increase the test reagent binding on the base and prevent the test reagent shaking off from the base. Preventing the test reagent shaking off from the base will maintain the amount of the test reagent with high test accuracy. Since the rough unit may be four lines formed multiple guiding channels located corresponding to the test reagent, the flow rate of the test sample received in the opening of the spacer can increase.

Other embodiments of the invention will appear to those skilled in the art from consideration of the specification and practice of the invention disclosed herein. It is intended that the specification and examples to be considered as exemplary only, with a true scope and spirit of the invention being indicated by the following claims.

What is claimed is:

1. An electrochemical biosensor strip, comprising:
a base;
an electrode system laid on the base, comprising a first end, a second end, at least three electrodes and a short circuit formed between two selected electrodes of the at least three electrodes;
a spacer covered on the electrode system to expose a portion of the first end for an electrical connection adapted to a meter, having an opening to expose a predetermined portion of the electrode system for applying a test reagent; and
a cover covered on the spacer.

2. The electrochemical biosensor strip as claimed in claim 1, wherein the electrode system comprises a silver layer laid on the base and a carbon layer laid on the silver layer.

3. The electrochemical biosensor strip as claimed in claim 2, wherein the silver layer has three electrodes and the carbon layer has four electrodes.

4. The electrochemical biosensor strip as claimed in claim 3, wherein the short circuit is formed between two selected electrodes of the four electrodes of the carbon layer.

5. The electrochemical biosensor strip as claimed in claim 2, wherein the carbon layer has five electrodes and the short circuit is formed between two selected electrodes thereof.

6. The electrochemical biosensor strip as claimed in claim 2, wherein the silver layer has a forked shape unit formed on the base and between the electrodes and the carbon layer has an arrowhead shape unit formed corresponding to the forked shape unit.

7. The electrochemical biosensor strip as claimed in claim 1, wherein one of the selected electrodes is a reference electrode.

8. The electrochemical biosensor strip as claimed in claim 1, wherein the cover has a channel with a hole formed corresponding to the opening of the spacer.

9. The electrochemical biosensor strip as claimed in claim 1, further comprising a rough unit formed on the base corresponding to the test reagent and adjacent to the electrode system.

10. The electrochemical biosensor strip as claimed in claim 1, wherein the electrodes on the first end of the electrode system are parallel.

11. The electrochemical biosensor strip as claimed in claim 1, wherein the opening of the spacer is formed in one side of the spacer.

12. The electrochemical biosensor strip as claimed in claim 1, further comprising an insulating substance laid on the electrode system.

13. The electrochemical biosensor strip as claimed in claim 12, wherein the opening of the spacer is formed in an end of the spacer and is T-shaped, and the insulating substance has an opening formed corresponding to the opening of the spacer.

14. An electrochemical biosensor strip comprising
a base;
an electrode system laid on the base;
a rough unit is formed on the base and near the electrode system;
a spacer covered on the electrode system to expose a portion of the electrode system for an electrical connection adapted to a meter, having an opening to expose a predetermined portion of the electrode system for applying of a test reagent; and
a cover covered on the spacer.

15. The electrochemical biosensor strip as claimed in claim 14, wherein the rough unit is a line or multiple lines.

16. The electrochemical biosensor strip as claimed in claim 14, wherein the rough unit is laid adjacent to the electrode system.

17. The electrochemical biosensor strip as claimed in claim 14, wherein the rough unit is prepared by electric conduction substance.

18. The electrochemical biosensor strip as claimed in claim 17, wherein the rough unit is prepared by carbon and separated from the electrode system.

19. The electrochemical biosensor strip as claimed in claim 14, wherein the rough unit is prepared by non-electric conduction substance.

20. The electrochemical biosensor strip as claimed in claim 14, wherein the electrode system comprises a silver layer laid on the base and a carbon layer laid on the silver layer.

21. The electrochemical biosensor strip as claimed in claim 14, wherein the electrode system comprises at least three electrodes, wherein a short circuit is formed between two selected electrodes of the three electrodes.

22. The electrochemical biosensor strip as claimed in claim 21, wherein the silver layer has a forked shape unit formed on the base and between the electrodes and the carbon layer has an arrowhead shape unit formed corresponding to the forked shape unit.

23. The electrochemical biosensor strip as claimed in claim 14, wherein the cover has a channel with a hole formed corresponding to the opening of the spacer.

24. The electrochemical biosensor strip as claimed in claim 23, wherein the opening of the spacer is formed in one side of the spacer.

25. The electrochemical biosensor strip as claimed in claim 14 further comprising an insulating substance laid on the electrode system and the opening of the spacer is formed in an end of the spacer and is T-shaped.

26. The electrochemical biosensor strip as claimed in claim 25, wherein the insulating substance has an opening formed corresponding to the opening of the spacer.
